(19) United States
(12) Patent Application Publication
(10) Pub. No.: US 2024/0115547 A1
(43) Pub. Date: Apr. 11, 2024

(54) PSYCHOTROPIC AGENTS AND USES THEREOF

(71) Applicant: LB PHARMACEUTICALS INC., New York, NY (US)

(72) Inventors: Andrew R. VAINO, New York, NY (US); Vincent T. GRATTAN, New York, NY (US); Zachary PRENSKY, New York, NY (US)

(21) Appl. No.: 18/516,614

(22) Filed: Nov. 21, 2023

Related U.S. Application Data
(63) Continuation of application No. PCT/US2022/029900, filed on May 18, 2022.
(60) Provisional application No. 63/189,905, filed on May 18, 2021.

(51) Int. Cl. A61K 31/40

(52) U.S. Cl. CPC A61K 31/40

(57) ABSTRACT

Disclosed herein are novel uses of amisulpride derivatives and pharmaceutical compositions thereof, alone or in combination with other CNS active agents to antagonize dopamine and/or serotonin (e.g., 5-HT2a, 5-HT7) and/or alpha 2 receptors in a subject. Amisulpride derivatives or pharmaceutical compositions thereof disclosed herein may be used, alone or in combination with other CNS active agents, for the treatment of one or more conditions responsive to modulation of dopamine and/or serotonin (e.g., 5-HT2a, 5-HT₇) and/or a 2 receptors in a subject. Amisulpride derivatives or pharmaceutical compositions thereof disclosed herein may be used alone or in combination with other CNS active agents for the treatment of one or more disorders associated with abnormal levels of dopamine and/or serotonin in the brain.

### CROSS-REFERENCE TO RELATED APPLICATIONS

This application is a continuation of International Application No. PCT/US2022/029900, filed May 18, 2022, which claims the benefit of U.S. Provisional Patent Application No. 63/189,905, filed May 18, 2021, both of which are incorporated by reference herein in its entirety.

### FIELD OF THE INVENTION

The present invention is generally in the field of pharmaceutical compositions and methods for the treatment of neuropsychiatric and/or psychological diseases or disorders.

### BACKGROUND

Schizophrenia is a chronic debilitating mental illness affecting about one percent of the population. The disease manifests in delusional behavior, dysfunctional thinking, agitated body movement, social withdrawal, and depression. Schizophrenia patients suffer a profoundly reduced quality of life, and are ten times more likely to commit suicide that the general population.

Dopamine (particularly D₂ and D₃) antagonists are well recognized as improving symptoms of schizophrenia, and have been used clinically as such for decades. In the past twenty years it has become recognized that treatment of schizophrenia, as with many mental illnesses, benefits from engaging multiple receptors including serotonergic and adrenergic. Despite, literally, dozens of approved drugs to treat schizophrenia the disease remains poorly treated in many patients. Side effects of current medications include: dyskinesia, akathisia, weight gain, mood disturbances, sexual dysfunction, sedation, orthostatic hypotension, hypersalivation, and (in some cases) arganulocytosis.

Amisulpride (4-amino-N-(((1-ethyl-2-pyrrolidinyl)methyl)-5-(ethylsulfonyl))-2-methoxybenzamide) is an antipsychotic patented in 1981. Amisulpride binds selectively to the human dopaminergic D₂ (Kᵢ 2.8 nM) and D₃ (Kᵢ 3.2 nM) receptor subtypes without any affinity for D₁, D₄ and D₅ receptor subtypes. Unlike classical and atypical neuroleptics, amisulpride displays low affinity for serotonin, alpha-adrenergic, histamine receptor subtypes, muscarinic receptors and sigma sites though it has also been demonstrated to bind 5-HT_{2B} and HT₇ₐ receptors with low double digit nM Kᵢ. This ability of amisulpride to bind 5-HT receptors is thought to result in amisulpride's ability to treat symptoms of depression (sometimes noted in schizophrenia patients), improve cognition, and may explain amisulpride's ability to treat negative symptoms of schizophrenia. Interestingly, compared to other antipsychotics, amisulpride is not noted to have any activity at the 5-HT₂ₐ receptor.

Despite the unique properties of amisulpride, the drug has low ability to cross the blood brain barrier (BBB) to interact with the receptors in the brain. In a 2014 study, passive diffusion of amisulpride across a PAMPA membrane (measured as Pₑ) was the lowest of 30 psychiatric drugs tested. Thus, dosing of amisulpride is high, typically 400 to 800 mg/d (though up to 1,200 mg/day is not uncommon). Such a high dose may cause adverse effects to the treated subjects.

### SUMMARY OF THE INVENTION

Provided herein are uses of amisulpride derivatives and pharmaceutical compositions thereof. In certain embodiments, the amisulpride derivatives disclosed herein are dopamine and/or serotonin antagonists. In certain embodiments, the amisulpride derivatives disclosed herein have improved membrane (e.g., BBB) permeability compared to amisulpride. In certain embodiments, the amisulpride derivatives can act as central nervous system (CNS) dopamine and/or serotonin antagonists. These amisulpride derivatives have structures of Formula IA, Formula IB or Formula IC disclosed herein, including pharmaceutically acceptable salts thereof, stereoisomers thereof (e.g., Formula IA-S, Formula IA-R, Formula IB-S, Formula IB-R, Formula IC-S, and Formula IC-R), or deuterated analogs of structures of Formula IA, Formula IB, Formula IC, Formula IA-S, Formula IA-R, Formula IB-S, Formula IB-R, Formula IC-S, or Formula IC-R.

Provided herein is a unit dose of an amisulpride derivative disclosed herein, the unit dose comprises a pharmaceutical composition comprising a therapeutically effective amount of the amisulpride derivative, and the therapeutically effective amount being about 10 mg to about 250 mg, about 10 mg to about 225 mg, about 10 mg to about 200 mg, about 10 mg to about 175 mg, about 10 mg to about 150 mg, about 10 mg to about 125 mg, about 10 mg to about 100 mg, about 10 mg to about 75 mg, about 10 mg to about 50 mg, about 10 mg to about 25 mg, about 25 mg to about 250 mg, about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 250 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 125 mg to about 250 mg, about 125 mg to about 225 mg, about 125 mg to about 200 mg, about 125 mg to about 175 mg, about 125 mg to about 150 mg, about 150 mg to about 250 mg, about 150 mg to about 225 mg, about 150 mg to about 200 mg, about 150 mg to about 175 mg, about 175 mg to about 250 mg, about 175 mg to about 225 mg, about 175 mg to about 200 mg, about 200 mg to about 250 mg, about 200 mg to about 225 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg.

Also provided herein are methods for delivering a dopamine and/or serotonin (e.g., 5-HT₂ₐ, 5-HT₇) and/or alpha-2 adrenergic (α2) receptor antagonist to the brain of a subject comprising administering to the subject a therapeutically effective amount of an amisulpride derivative disclosed herein or a pharmaceutical composition thereof; and the dopamine and/or serotonin and/or α2 receptor antagonist level in the brain being higher than administering to the subject amisulpride at a comparable dose. In certain embodiments, the method comprises administering to the subject one, two, three, or four unit doses of an amisulpride derivative disclosed herein. In certain embodiments, the method comprises administering to the subject one, two, three, or four unit doses of an amisulpride derivative disclosed herein once a day, once every two days, once every three days, once every four days, once every five days, once every six days, or once a week. In certain embodiments, the unit dose is 50 mg, 75 mg, or 100 mg. In certain embodiments, the method further comprising adjusting the dose of the amisulpride derivative to accomplish a striatal dopamine RO % or an average dopamine (e.g., D₂/D₃) RO % of caudate and putamen measured from a treated subject to about 60% to about 80%, about 50% to about 85%, or about 40% to about 90%.

Also provided herein are methods for antagonizing dopamine and/or serotonin (e.g., 5-HT2a, 5-HT₇) and/or α2 receptor in a subject comprising administering to a subject a therapeutically effective amount of an amisulpride derivative disclosed herein or pharmaceutical compositions thereof, either individually or in combination with other CNS active agents. In certain embodiments, the method comprises administering to the subject one, two, three, or four unit doses of an amisulpride derivative disclosed herein. In certain embodiments, the method comprises administering to the subject one, two, three, or four unit doses of an amisulpride derivative disclosed herein once a day, once every two days, once every three days, once every four days, once every five days, once every six days, or once a week. In certain embodiments, the unit dose is 50 mg, 75 mg, or 100 mg. In certain embodiments, the method further comprising adjusting the dose of the amisulpride derivative to accomplish a striatal dopamine RO % or an average dopamine (e.g., D₂/D₃) RO % of caudate and putamen measured from a treated subject to about 60% to about 80%, about 50% to about 85%, or about 40% to about 90%.

Also provided herein are methods for treating one or more conditions responsive to modulation of dopamine and/or serotonin (e.g., 5-HT2a, 5-HT₇) and/or α2 receptor in a subject comprising administering to a subject a therapeutically effective amount of an amisulpride derivative disclosed herein or pharmaceutical compositions thereof, either individually or in combination with other CNS active agents. In certain embodiments, the method comprises administering to the subject one, two, three, or four unit doses of an amisulpride derivative disclosed herein. In certain embodiments, the method comprises administering to the subject one, two, three, or four unit doses of an amisulpride derivative disclosed herein once a day, once every two days, once every three days, once every four days, once every five days, once every six days, or once a week. In certain embodiments, the unit dose is 50 mg, 75 mg, or 100 mg. In certain embodiments, the method further comprising adjusting the dose of the amisulpride derivative to accomplish a striatal dopamine RO % or an average dopamine (e.g., D₂/D₃) RO % of caudate and putamen measured from a treated subject to about 60% to about 80%, about 50% to about 85%, or about 40% to about 90%.

Also provided herein are methods for treating one or more disorders associated with an abnormality in levels of dopamine and/or serotonin in the brain, comprising administering to a subject a therapeutically effective amount of an amisulpride derivative disclosed herein or pharmaceutical compositions thereof, either individually or in combination with other CNS active agents. In certain embodiments, the method comprises administering to the subject one, two, three, or four unit doses of an amisulpride derivative disclosed herein. In certain embodiments, the method comprises administering to the subject one, two, three, or four unit doses of an amisulpride derivative disclosed herein once a day, once every two days, once every three days, once every four days, once every five days, once every six days, or once a week. In certain embodiments, the unit dose is 50 mg, 75 mg, or 100 mg. In certain embodiments, the method further comprising adjusting the dose of the amisulpride derivative to accomplish a striatal dopamine RO % or an average dopamine (e.g., D2/D3) RO % of caudate and putamen measured from a treated subject to about 60% to about 80%, about 50% to about 85%, or about 40% to about 90%.

Examples of the conditions responsive to modulation of dopamine and/or serotonin (e.g., 5-HT2a, 5-HT7) and/or α2 receptor and/or and the disorders associated with abnormality in levels of dopamine and/or serotonin in the brain include, e.g., without limitation, mental illnesses. Examples of the mental illnesses include, without limitation, schizophrenia, symptoms of schizophrenia, schizoaffective disorder, bipolar disorder, depression, obsessive-compulsive disorder, Parkinson's psychosis, Alzheimer's psychosis, oppositional defiant disorder, aggression, suicidality, hostility, personality disorders, chronic fatigue syndrome, predominantly negative symptoms of schizophrenia, Charles Bonnet Syndrome, autism, and Tourette's disorder.

### BRIEF DESCRIPTION OF THE DRAWINGS

FIG. 1A: Dopamine (e.g., D2/D3) % RO for 50 mg LB-102 oral administration to human subjects (n=4).

FIG. 1B: Dopamine (e.g., D2/D3) % RO for 100 mg LB-102 oral administration to human subjects (n=3).

FIG. 2: Plasma concentration of LB-102 (diamond), amisulpride (square), and total benzamide (triangle) after 50 mg LB-102 oral administration to human subjects (n=4).

FIG. 3: Plasma concentration as a function of time for single oral administration of LB-102 to human subjects at 10 mg (larger cross), 50 mg (triangle), 100 mg (square), 150 mg (smaller cross), and 200 mg (diamond), respectively.

FIG. 4A: PK profile of an administration of 50 mg amisulpride previously published.

FIG. 4B: PK profile of a single oral administration of LB-102 (50 mg) to human subjects.

FIG. 5A: Average dopamine (e.g., D2/D3) RO % in caudate and putamen of subjects treated with LB-102 and PK analysis of same after a single oral administration of 50 mg LB-102.

FIG. 5B: Average dopamine (e.g., D2/D3) RO % in caudate and putamen of subjects treated with LB-102 and PK analysis of same after a single oral administration of 75 mg LB-102.

FIG. 5C: Average dopamine (e.g., D2/D3) RO % in caudate and putamen of subjects treated with LB-102 and PK analysis of same after a single oral administration of 100 mg LB-102.

FIG. 6A: Average dopamine (e.g., D2/D3) RO % in caudate and putamen of subjects treated with LB-102 and PK analysis of same after the LB-102 administration on day 4 of oral administration of 50 mg LB-102/day.

FIG. 6B: Average dopamine (e.g., D2/D3) RO % in caudate and putamen of subjects treated with LB-102 and PK analysis of same after the LB-102 administration on day 4 of oral administration of 100 mg LB-102/day

### DETAILED DESCRIPTION OF THE INVENTION

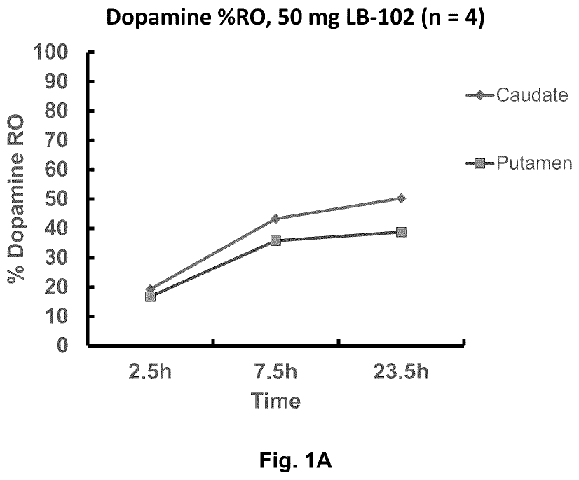

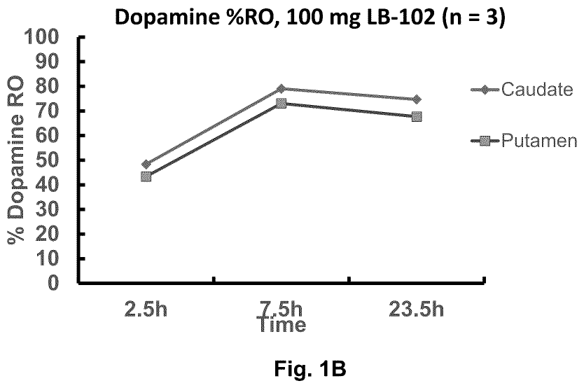

Dopamine receptor occupancy (RO) is a well-established marker of antipsychotic efficacy: 60 to 75% RO correlates to meaningful improvements in PANSS scores in schizophrenia patients [Pani, L., Pira, L., Marchese, G., 2007. “Antipsychotic efficacy: Relationship to optimal D2-receptor occupancy”, European Psychiatry, 22, 267-275.]. As disclosed herein, amisulpride derivatives disclosed herein (also referred to as 4-amino substituted derivatives of amisulpride, -amino amisulpride derivatives and 4-amino substituted amisulpride derivatives) achieved desired dopamine (e.g., D2/D3) RO in caudate and putamen in the cerebrum of human subjects at a dosage significantly lower than the dose of amisulpride required to reach comparable dopamine (e.g., D₂/D₃) RO. See, e.g., Example 1, a single oral dose of 50 mg and 100 g LB102 demonstrated dopamine D₂/D₃RO about 50% (FIG. 1A) and 75% (FIG. 1B) in caudate/putamen in the cerebrum of human subjects, respectively, while 75% dopamine (e.g., D₂/D₃) RO required amisulpride of a dose of more than 400 mg. [Meisenzahl, E. M., Schmitt, G., Grunder, G., Dresel, S., Frodl, T., la Fougère, C., Scheuerecker, J. Schwarz, M., Strauss, J. Hahn, K., and Möller, H.-J., 2008, “Striatal D₂/D₃ Receptor Occupancy, Clinical Response and Side Effects with Amisulpride: An Iodine-123-Iodobenzamide SPET Study, Pharmacopsychiatry, 41, 169-175.]

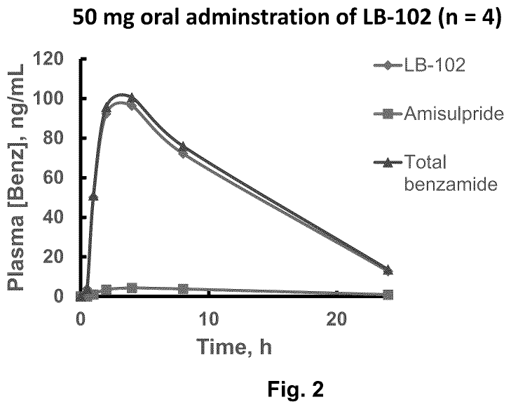

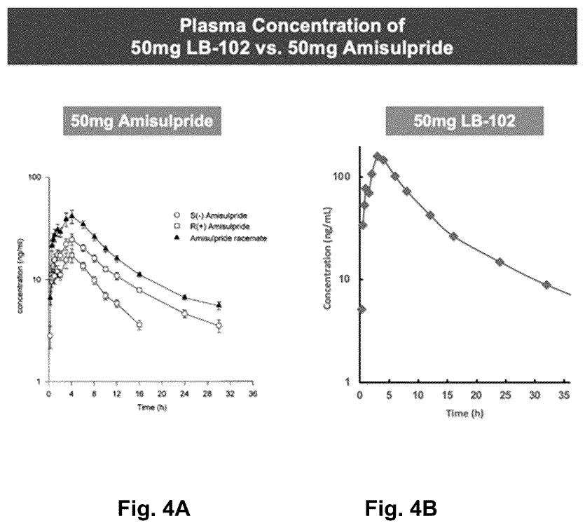

Plasma exposure of LB-102 in human subjects was markedly above expectations compared to animal models and published data on amisulpride. In preclinical animal models LB-102 was extensively, up to 50%, demethylated to amisulpride. However, as shown in Example 2, metabolism of LB-102 to amisulpride was minimal (<3%) in human (FIG. 2). Furthermore, see, e.g., Example 2, oral administration of 50 mg LB102 to human subjects (FIG. 4B) showed an AUC (1,595 ngh/mL) that was about 2.5 times of the AUC (603 ngh/mL) obtained by administration of 50 mg amisulpride (FIG. 4A) [M. P. Curran and C. M. Perry “Amisulpride: a review of its use in the management of schizophrenia”, Drugs, 2001, 61, 2132-2150.].

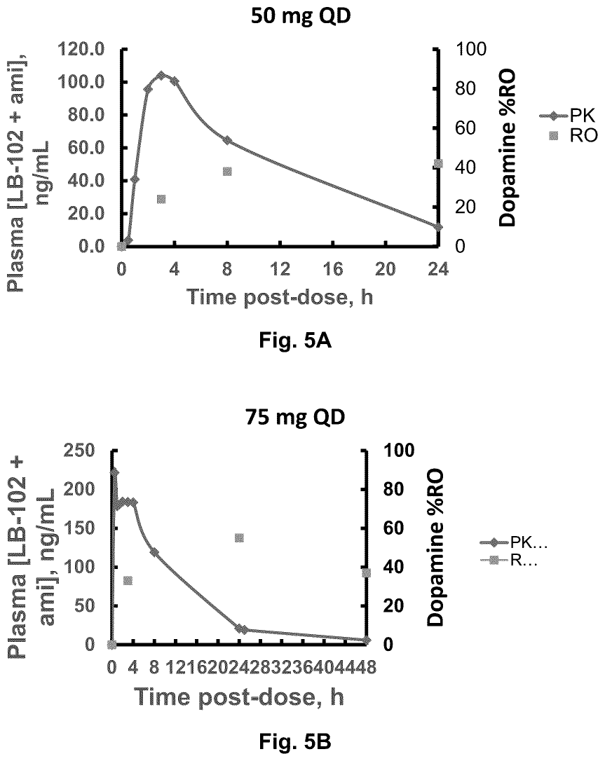

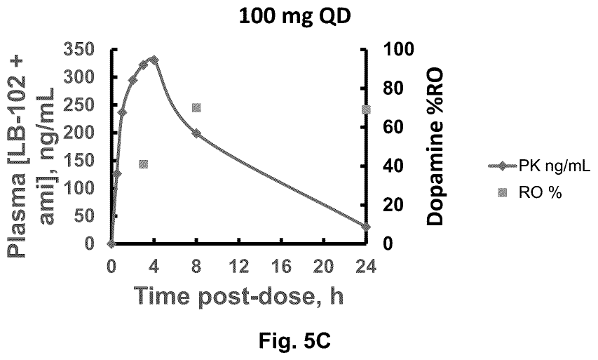

Unexpectedly, average dopamine (e.g., D2/D3) RO % in caudate, putamen, and thalamus of subjects treated with LB-102 (50 mg SS; and 100 mg SS) significantly stabilized at least from Day 4 since the treatment began (Example 3, Tables 3-E and 3-F summarizing data after the dosing on Day 4; and Tables 3-B and 3-D summarizing data after the first dose which was administered on Day 1), while the profiles of the plasma concentration of LB102 and amisulpride were similar after dosing on Day 1 (FIGS. 5A&5C) and Day 4 (FIGS. 5D&5E). It was surprising to observe that higher plasma concentration of LB102 and amisulpride did not lead to higher dopamine RO %.

Provided herein are uses of amisulpride derivatives and pharmaceutical compositions thereof. In certain embodiments, the amisulpride derivatives disclosed herein are dopamine and/or serotonin antagonists. In certain embodiments, the amisulpride derivatives disclosed herein have improved membrane (e.g., BBB) permeability compared to amisulpride. In certain embodiments, the amisulpride derivatives can act as central nervous system (CNS) dopamine and/or serotonin antagonists. In certain embodiments, the amisulpride derivatives disclosed herein more selectively bind to dopamine D2 and/or D3 receptor over dopamine D₁, D₄ and/or D₅ receptor. In certain embodiments, the amisulpride derivatives disclosed herein are capable of interacting dopamine and/or serotonin and/or α2 receptors in CNS.

These amisulpride derivatives have structures of Formula IA, Formula IB or Formula IC disclosed herein, including pharmaceutically acceptable salts thereof, stereoisomers thereof (e.g., Formula IA-S, Formula IA-R, Formula IB-S, Formula IB-R, Formula IC-S, and Formula IC-R), or deuterated analogs of structures of Formula IA, Formula IB, Formula IC, Formula IA-S, Formula IA-R, Formula IB-S, Formula IB-R, Formula IC-S, or Formula IC-R.

In certain embodiments, deuterated analogs of a compound has one or more hydrogens of the compound replaced by deuterium. In certain embodiments, one or more deuteriums in the deuterated analog are present in at least 100 times the natural abundance level.

Provided herein are pharmaceutical compositions comprising one or more of the amisulpride derivatives disclosed herein and a pharmaceutically acceptable carrier. In certain embodiments, the one or more of the amisulpride derivatives the pharmaceutical compositions comprise are substantially enantiomerically pure, and such pharmaceutical compositions are also referred to as substantially enantiomerically pure pharmaceutical compositions. In certain embodiments, the term “substantially enantiomerically pure” means enantiomerical purity of about 50% or higher, about 60% or higher, about 70% or higher, about 80% or higher, about 90% or higher, about 95% or higher, or about 98% or higher.

Provided herein is a unit dose of an amisulpride derivative disclosed herein, the unit dose comprises a pharmaceutical composition comprising a therapeutically effective amount of the amisulpride derivative, and the therapeutically effective amount being about 10 mg to about 250 mg, about 10 mg to about 225 mg, about 10 mg to about 200 mg, about 10 mg to about 175 mg, about 10 mg to about 150 mg, about 10 mg to about 125 mg, about 10 mg to about 100 mg, about 10 mg to about 75 mg, about 10 mg to about 50 mg, about 10 mg to about 25 mg, about 25 mg to about 250 mg, about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 250 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 125 mg to about 250 mg, about 125 mg to about 225 mg, about 125 mg to about 200 mg, about 125 mg to about 175 mg, about 125 mg to about 150 mg, about 150 mg to about 250 mg, about 150 mg to about 225 mg, about 150 mg to about 200 mg, about 150 mg to about 175 mg, about 175 mg to about 250 mg, about 175 mg to about 225 mg, about 175 mg to about 200 mg, about 200 mg to about 250 mg, about 200 mg to about 225 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg.

Also provided herein are methods for delivering a dopamine and/or serotonin (e.g., 5-HT2a, 5-HT₇) and/or alpha-2 adrenergic (α2) receptor antagonist to the brain of a subject comprising administering to the subject a therapeutically effective amount of an amisulpride derivative disclosed herein or a pharmaceutical composition thereof; and the dopamine and/or serotonin and/or α2 receptor antagonist level in the brain being higher than administering to the subject amisulpride at a comparable dose. In certain embodiments, the method comprises administering to the subject one, two, three, or four unit doses of an amisulpride derivative disclosed herein. In certain embodiments, the method comprises administering to the subject one, two, three, or four unit doses of an amisulpride derivative disclosed herein once a day, once every two days, once every three days, once every four days, once every five days, once every six days, or once a week. In certain embodiments, the unit dose is 50 mg, 75 mg, or 100 mg. In certain embodiments, the striatal dopamine RO % or average dopamine (e.g., D2/D3) RO % of caudate and putamen measured from a treated subject is about 60% to about 80%, about 50% to about 85%, or about 40% to about 90%. In certain embodiments, the method further comprising adjusting the dose of the amisulpride derivative to accomplish a striatal dopamine RO % or an average dopamine (e.g., D2/D3) RO % of caudate and putamen measured from a treated subject to about 60% to about 80%, about 50% to about 85%, or about 40% to about 90%.

Also provided herein are methods for antagonizing dopamine and/or serotonin (e.g., 5-HT2a, 5-HT7) and/or α2 receptor in a subject comprising administering to a subject a therapeutically effective amount of an amisulpride derivative disclosed herein or pharmaceutical compositions thereof, either individually or in combination with other CNS active agents. In certain embodiments, the method comprises administering to the subject one, two, three, or four unit doses of an amisulpride derivative disclosed herein. In certain embodiments, the method comprises administering to the subject one, two, three, or four unit doses of an amisulpride derivative disclosed herein once a day, once every two days, once every three days, once every four days, once every five days, once every six days, or once a week. In certain embodiments, the unit dose is 50 mg, 75 mg, or 100 mg. In certain embodiments, the method further comprising adjusting the dose of the amisulpride derivative to accomplish a striatal dopamine RO % or an average dopamine (e.g., D2/D3) RO % of caudate and putamen measured from a treated subject to about 60% to about 80%, about 50% to about 85%, or about 40% to about 90%.

Also provided herein are methods for treating one or more conditions responsive to modulation of dopamine and/or serotonin (e.g., 5-HT2a, 5-HT7) and/or α2 receptor in a subject comprising administering to a subject a therapeutically effective amount of an amisulpride derivative disclosed herein or pharmaceutical compositions thereof, either individually or in combination with other CNS active agents. In certain embodiments, the method comprises administering to the subject one, two, three, or four unit doses of an amisulpride derivative disclosed herein. In certain embodiments, the method comprises administering to the subject one, two, three, or four unit doses of an amisulpride derivative disclosed herein once a day, once every two days, once every three days, once every four days, once every five days, once every six days, or once a week. In certain embodiments, the unit dose is 50 mg, 75 mg, or 100 mg. In certain embodiments, the method further comprising adjusting the dose of the amisulpride derivative to accomplish a striatal dopamine RO % or an average dopamine (e.g., D2/D3) RO % of caudate and putamen measured from a treated subject to about 60% to about 80%, about 50% to about 85%, or about 40% to about 90%.

Also provided herein are methods for treating one or more disorders associated with an abnormality in levels of dopamine and/or serotonin in the brain, comprising administering to a subject a therapeutically effective amount of an amisulpride derivative disclosed herein or pharmaceutical compositions thereof, either individually or in combination with other CNS active agents. In certain embodiments, the method comprises administering to the subject one, two, three, or four unit doses of an amisulpride derivative disclosed herein. In certain embodiments, the method comprises administering to the subject one, two, three, or four unit doses of an amisulpride derivative disclosed herein once a day, once every two days, once every three days, once every four days, once every five days, once every six days, or once a week. In certain embodiments, the unit dose is 50 mg, 75 mg, or 100 mg. In certain embodiments, the method further comprising adjusting the dose of the amisulpride derivative to accomplish a striatal dopamine RO % or an average dopamine (e.g., D2/D3) RO % of caudate and putamen measured from a treated subject to about 60% to about 80%, about 50% to about 85%, or about 40% to about 90%.

In certain embodiments of the methods disclosed herein, the method further comprising:
a) administering a first unit dose of the amisulpride derivative to the subject once a day for one day, two days, three days, four days, five days, six days, or a week;
b) obtaining a first average dopamine RO % of caudate and putamen of the subject;
c) administering to the subject a second dose of the amisulpride derivative once a day for one day, two days, three days, four days, five days, six days, or a week if a first striatal dopamine RO % or a first average dopamine (e.g., D2/D3) RO % of caudate and putamen of the subject is outside of a predetermined range of about 60% to about 80%, about 50% to about 85%, or about 40% to about 90%;
d) obtaining a second striatal dopamine RO % or a second average dopamine (e.g., D2/D3) RO % of caudate and putamen of the subject; and
e) repeat steps c) and d) until the striatal dopamine RO % or average dopamine (e.g., D2/D3) RO % of caudate and putamen of the subject falls within the predetermined range (e.g., about 60% to about 80%, about 50% to about 85%, or about 40% to about 90%).

Examples of the conditions responsive to modulation of dopamine and/or serotonin (e.g., 5-HT2a, 5-HT₇) and/or a2 receptor and/or and the disorders associated with abnormality in levels of dopamine and/or serotonin in the brain include, e.g., without limitation, mental illnesses. Examples of the mental illnesses include, without limitation, schizophrenia, symptoms of schizophrenia, schizoaffective disorder, bipolar disorder, depression, obsessive-compulsive disorder, Parkinson's psychosis, Alzheimer's psychosis, oppositional defiant disorder, aggression, suicidality, hostility, personality disorders, chronic fatigue syndrome, predominantly negative symptoms of schizophrenia, Charles Bonnet Syndrome, autism, and Tourette's disorder.

4-Amino Substituted Amisulpride Derivatives

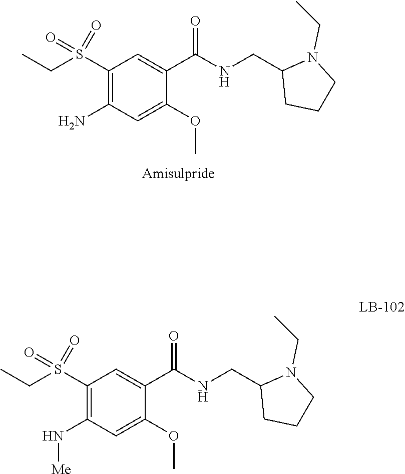 [SMILES: CCN1CCCC1CNC(=O)c1cc(S(=O)(=O)CC)c(N)cc1OC ; CCN1CCCC1CNC(=O)c1cc(S(=O)(=O)CC)c(NC)cc1OC]

In certain embodiments, the amisulpride derivative is a 4-amino substituted derivative of amisulpride having a structure of Formula IA:

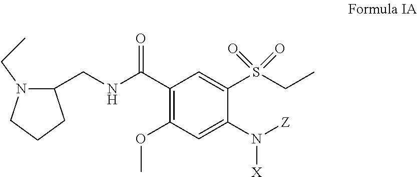

including pharmaceutically acceptable salts and stereoisomers thereof, and X and Z are the same or different and independently selected from the group consisting of hydrogen, alkyl (either branched or unbranched, such as methyl, ethyl, n-propyl, i-propyl, n-butyl, and s-butyl), alkenyl (either branched or unbranched, such as methyl, ethyl, n-propyl, i-propyl, n-butyl, and s-butyl), alkynyl (either branched or unbranched, such as methyl, ethyl, n-propyl, propyl, n-butyl, and s-butyl), cycloalkyl (e.g., cyclopropyl, cyclobutyl, cyclopentyl, and cyclohexyl), cycloalkylalkyl (e.g., cyclopropylmethyl, cyclobutylethyl, and cyclopentylethyl), heterocyclyl, heterocyclylalkyl, aryl (e.g., phenyl, naphthyl, tetrahydronapthyl, indanyl, and biphenyl), arylalkyl (e.g., —CH₂C₆H₅, and —C₂H₅C₆H₅), heteroarylalkyl (e.g., —CH₂C₆H₄N, and —C₂H₅C₆H₄N), and heteroaryl with one or two or three or more hetero ring atoms (such as pyridine, pyrrole, furan, thiophene, or pyrimidine), optionally the alkyl, alkenyl, alkynyl, cycloalkyl, cycloalkylalkyl, heterocyclyl, heterocyclylalkyl, aryl, arylalkyl, heteroarylalkyl, and heteroaryl groups are further substituted with one or more substitution groups selected from the group consisting of halogens such as chlorine, bromine and fluorine, amines, hydroxy groups, carboxylic acids, nitro groups, carbonyl and other alkyl and aryl groups as defined herein; with the proviso that at least one of X and Z is not hydrogen.

In certain embodiments, the 4-amino substituted derivative of amisulpride is a stereoisomer having a structure of Formula IA-R:

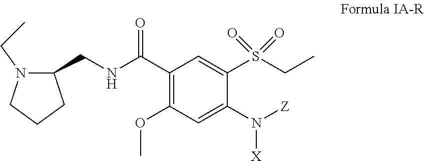

including pharmaceutically acceptable salts thereof, and X and Z are defined the same as above with respect to Formula IA.

In certain embodiments, the 4-amino substituted derivative of amisulpride is a stereoisomer having a structure of Formula IA-S:

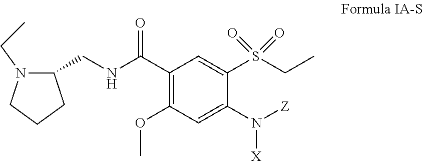

including pharmaceutically acceptable salts thereof, and X and Z are defined the same as above with respect to Formula IA.

In certain embodiments, the amisulpride derivative is a 4-amino substituted derivative of amisulpride having a structure of Formula IB:

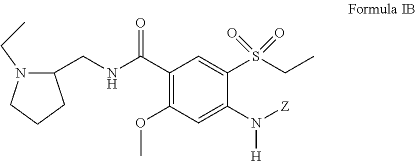

including pharmaceutically acceptable salts and stereoisomers thereof, and Z is defined the same as above with respect to Formula IA with the proviso that Z is not H.

In certain embodiments, the 4-amino substituted derivative of amisulpride is a stereoisomer having a structure of Formula IB-R:

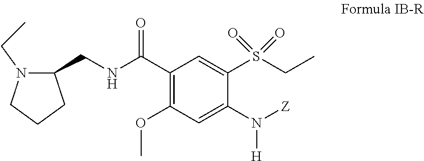

including pharmaceutically acceptable salts thereof, and Z is defined the same as above with respect to Formula IA with the proviso that Z is not H.

In certain embodiments, the 4-amino substituted derivative of amisulpride is a stereoisomer having a structure of Formula IB-S:

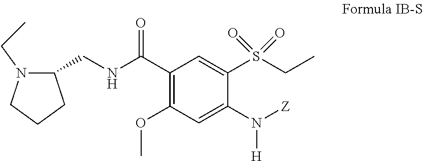

including pharmaceutically acceptable salts thereof, and Z is defined the same as above with respect to Formula IA with the proviso that Z is not H.

In certain embodiments, the amisulpride derivative has a structure of Formula IC:

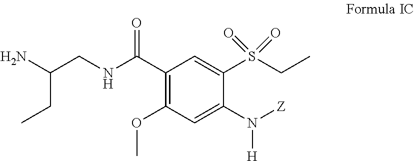

including pharmaceutically acceptable salts and stereoisomers thereof, and Z is defined the same as above with respect to Formula IA with the proviso that Z is not H.

In certain embodiments, the amisulpride derivative is a stereoisomer having a structure of Formula IC-R:

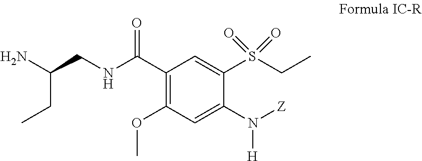

including pharmaceutically acceptable salts thereof, and Z is defined the same as above with respect to Formula IA with the proviso that Z is not H.

In certain embodiments, the amisulpride derivative is a stereoisomer having a structure of Formula IC-S:

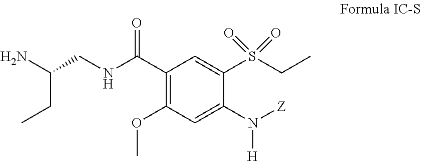

including pharmaceutically acceptable salts thereof, and Z is defined the same as above with respect to Formula IA with the proviso that Z is not H.

As used herein, the singular for “a,” “an,” and “the” include plural references unless the context clearly dictates otherwise. For example, the term “a cell” includes a plurality of cells, including mixtures thereof. Similarly, use of “a compound” for treatment of preparation of medicaments as described herein contemplates using one or more compounds of the invention for such treatment or preparation unless the context clearly dictates otherwise.

As used herein, the term “comprising” is intended to mean that the compositions and methods include the recited elements, but not excluding others. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the composition of this invention. Embodiments defined by each of the transitional terms are within the scope of this invention.

The term “alkyl” refers to a straight or branched hydrocarbon chain radical consisting solely of carbon and hydrogen atoms, containing no unsaturation. Unless otherwise specified, the term “alkyl” refers to a group having one, two, three, four, five, six, seven, or eight carbon atoms (for example, one to six carbon atoms, or one to four carbon atoms), and which is attached to the rest of the molecule by a single bond. Examples of alkyl groups include, but are not limited to, methyl, ethyl, n-propyl, propyl, n-butyl, t-butyl, s-butyl, n-pentyl, and s-pentyl.

The term “alkenyl” refers to an aliphatic hydrocarbon group containing a carbon-carbon double bond and which may be a straight or branched or branched chain. Unless otherwise specified, the term “alkenyl” refers to a group having 2, 3, 4, 5, 6, 7, 8, 9, or 10 carbon atoms, e.g., ethenyl, 1-propenyl, 2-propenyl (allyl), iso-propenyl, 2-methyl-1-propenyl, 1-butenyl, and 2-butenyl.

The term “alkynyl” refers to a straight or branched chain hydrocarbyl radical having at least one carbon-carbon triple bond. Unless otherwise specified, the term “alkynyl” refers to a group having in the range of 2, 3, 4, 5, 6, 7, 8, 9, 10, 11, or 12 carbon atoms (for instance, 2 to 10, 2 to 10 carbon atoms), e.g., ethynyl, propynyl, and butnyl.

The term “cycloalkyl” denotes a non-aromatic mono or multicyclic ring system of 3, 4, 5, 6, 7, 8, 9, 10, 11, or 12 carbon atoms such as cyclopropyl, cyclobutyl, cyclopentyl, and cyclohexyl.

The term “cycloalkylalkyl” refers to a cycloalkyl group as defined above directly bonded to an alkyl group as defined above.

The term “aryl” refers to a mono- or multi-cyclic aromatic radical having in the range of 6 up to 20 carbon atoms such as phenyl, naphthyl, tetrahydronapthyl, indanyl, and biphenyl.

The term “arylalkyl” refers to an aryl group as defined above directly bonded to an alkyl group as defined above, e.g., —CH₂C₆H₅, and —C₂H₅C₆H₅.

The term “heterocyclyl” refers to a non-aromatic 3 to 15 member ring radical which, consists of carbon atoms and at least one heteroatom selected from the group consisting of nitrogen, phosphorus, oxygen and sulfur. The heterocyclic ring radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized.

The term “heterocyclylalkyl” refers to a heterocyclyl group as defined above directly bonded to an alkyl group as defined above.

The term “heteroaryl” refers to an optionally substituted 5-14 member aromatic ring having one or more hetero ring atoms selected from the group consisting of N, O, and S as ring atoms. The heteroaryl may be a mono-, bi- or tricyclic ring system. Examples of such heteroaryl ring radicals includes, but are not limited to, oxazolyl, thiazolyl imidazolyl, pyrrolyl, furanyl, pyridinyl, pyrimidinyl, pyrazinyl, benzofuranyl, indolyl, benzothiazolyl, benzoxazolyl, carbazolyl, quinolyl, and isoquinolyl.

The term “heteroarylalkyl” refers to an heteroaryl group as defined above directly bonded to an alkyl group as defined above, e.g., —CH₂C₆H₄N, and —C₂H₅C₆H₄N.

The term “subject” refers to a mammal, such as a domestic pet (for example, a dog or cat), or human. In certain embodiments, the subject is a human.

The phrase “effective amount” refers to the amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

The term “salt” used herein is not limited as long as the salt is formed with a compound of the amisulpride derivatives and is pharmaceutically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, and the like. In addition, hydrochloride salt, sulfate salt, methanesulfonate salt, acetate salt and the like are preferred as “pharmacologically acceptable salt” of the amisulpride derivatives disclosed herein.

Isomers of the amisulpride derivatives disclosed herein (e.g., geometric isomers, optical isomers, rotamers, tautomers, and the like) can be purified using general separation means, including for example recrystallization, optical resolution such as diastereomeric salt method, enzyme fractionation method, various chromatographies (for instance, thin layer chromatography, column chromatography, glass chromatography and the like) into a single isomer.

Pharmaceutical Formulations and Routes of Administration

The amisulpride derivatives disclosed herein may be administered by a variety of routes including orally and by injection (e.g. subcutaneously, intravenously, and intraperitoneally). The amisulpride derivatives disclosed herein may be formulated into a pharmaceutical composition for use in the disclosed methods. Such compositions are prepared in accordance with acceptable pharmaceutical procedures such as described in Remington's Pharmaceutical Sciences, 17th edition, ed. Alfonso R. Gennaro, Mack Publishing Company, Eaton, Pa. (1985), which is incorporated herein by reference.

The amisulpride derivatives disclosed herein may be administered orally in the form of a solid or liquid dosage form. In both, the amisulpride derivatives disclosed herein compound may be coated in a material to protect it from the action of acids and other natural conditions which may inactivate the compound. The amisulpride derivatives disclosed herein may be formulated as aqueous solutions, liquid dispersions, (ingestible) tablets, buccal tablets, troches, capsules, elixirs, suspensions, syrups, and wafers. The oral dosage forms may include excipients known in the art, such as binders, disintegrating agents, flavorants, antioxidants, and preservatives. Liquid dosage forms may include diluents such as saline or an aqueous buffer.

The amisulpride derivatives disclosed herein may also be administered by injection. Formulations suitable for injection may include sterile aqueous solutions (where water soluble) or dispersions, and sterile powders for the extemporaneous preparation of sterile injectable solutions or dispersions. The pharmaceutical composition may be sterile and be fluid to the extent that easy syringability exists. It may be stable under the conditions of manufacture and storage and be preserved against the contaminating action of microorganisms such as bacteria and fungi. The pharmaceutically acceptable carrier can be a solvent or dispersion medium containing, for example, water, ethanol, polyol (such as, glycerol, propylene glycol, and liquid polyethylene glycol), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, and ascorbic acid. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the methods of preparation include vacuum drying and freeze-drying which yields a powder of the active ingredient (i.e., the therapeutic compound) plus any additional desired ingredient from a previously sterile-filtered solution thereof.

The actual dosage amount of the compound administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject.

In one embodiment, a human subject is administered the daily doses of from about 0.01 mg/kg to about 100 mg/kg.

Single or multiple doses of the amisulpride derivatives are contemplated. Desired time intervals for delivery of multiple doses can be determined by one of ordinary skill in the art employing no more than routine experimentation. As an example, subjects may be administered two doses daily at approximately 12 hour intervals. In some embodiments, the amisulpride derivatives are administered once a day.

The amisulpride derivatives disclosed herein or pharmaceutical compositions thereof may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months. In other embodiments, the invention provides that the amisulpride derivatives disclosed herein or pharmaceutical compositions thereof agent(s) may be taken orally and that the timing of which is or is not dependent upon food intake. Thus, for example, the agent can be taken every morning and/or every evening, regardless of when the subject has eaten or will eat.

Combination Therapy

In addition to being used as a monotherapy, the amisulpride derivatives disclosed herein or pharmaceutical compositions thereof may also find use in combination therapies. Effective combination therapy may be achieved with a single pharmaceutical composition or pharmacological formulation that includes both agents, or with two distinct pharmaceutical compositions or pharmacological formulations, administered at the same time, wherein one composition includes a compound of this invention, and the other includes the second agent(s). Alternatively, the therapy may precede or follow the other agent treatment by intervals ranging from minutes to months.

The additional agent or agents may be selected from any agent or agents useful for treating a psychological disorder, for example any agent or agent and/or a25 useful for treating an imbalance of dopamine, serotonin, histamine, or glutamate. In one embodiment, the additional agent or agent is useful in improving psychological function, e.g., an antipsychotic, such as quetiapine, geodon, zyprexa, latuda, olanzapine, risperidone, iloperidone, ziprasidone, clozapine, haloperidol, chlorpromazine, citrlopram, escitalopram, paroxetine, fluoxetine, fluvoxamine, sertraline, desvenlafaxine, duloxetine, milnacipran, venlafaxine, vilazodone, and combinations thereof.

Having described the invention with reference to the embodiments and illustrative examples, those in the art may appreciate modifications to the invention as described and illustrated that do not depart from the spirit and scope of the invention as disclosed in the specification. The examples are set forth to aid in understanding the invention but are not intended to, and should not be construed to limit its scope in any way. The examples do not include detailed descriptions of conventional methods. Such methods are well known to those of ordinary skill in the art and are described in numerous publications. Further, all references cited above and in the examples below are hereby incorporated by reference in their entirety, as if fully set forth herein.

### EXAMPLES

### Example 1: D₂/D₃ RO Analysis of Administration of LB-102 to Healthy Human Subjects

Healthy volunteers were dosed orally with either 50 mg (n=4) or 100 mg (n=3) LB-102 and dynamic ¹¹C raclopride PET scans were obtained at baseline, 2.5, 7.5, and 23.5 h after LB-102 administration to provide D₂/D₃ RO (Tables 1-A and 1-B) See also FIGS. 1A (50 mg) and 1B (100 mg) which showed D₂/D₃ RO of caudate (diamond) and putamen (square).

TABLE 1-A
Caudate % RO

| LB102 (mg) | 2.5 h after admin | 7.5 h after admin | 23.5 h after admin. |
|---|---|---|---|
| 100 | 48 | 77 | 74 |
| 50 | 26 | 43 | 50 |

TABLE 1-B
Putamen % RO

| LB102 (mg) | 2.5 h after admin | 7.5 h after admin | 23.5 h after admin. |
|---|---|---|---|
| 50 | 22 | 36 | 39 |
| 100 | 42 | 71 | 67 |

### Example 2: PK Analysis of Administration of LB-102 to Healthy Human Subjects

I) Plasma concentration of LB-102 (diamond), amisulpride (square), and total benzamide (triangle) after 50 mg LB-102 oral administration to human subjects were obtained and shown in FIG. 2 (n=4).

II) Study Design and Subjects

This study was conducted at a single site in compliance with all Institutional Review Board regulations. All local regulations and Good Clinical Practices were observed.

Healthy females and males between 18 and 55 years of age with BMI≥18 and ≤30 kg/m2 were enrolled in the study. Exclusion criteria included: history or presence of psychiatric disorder, drug or alcohol abuse, history of QT prolongation or dysrhythmia, fasting blood glucose level of 126 mg/dL, or a known allergy to the drug or its metabolites. The study was planned as a two part study. Part A was comprised of 5 single ascending dose groups, each of 8 subjects. Part B was comprised of 3 multiple ascending doses, two doses a day for 7 days (13 doses in total) each of 8 subjects. All subjects were randomized 3:1 drug:placebo. The primary endpoint of the study was safety, with pharmacokinetics as a secondary objective.

Demographics

A total of 64 healthy volunteers were enrolled in this study, and demographic data are summarized in Table 2-A. There were numerical differences between treatment and placebo groups in mean age and female/male ratio as well as a preponderance of African American/Black subjects: BMI was well-matched.

TABLE 2-A
Demographic characteristics of enrolled subjects.

| | Part A (SAD) | | | | | Part B (MAD) | | | | |
|---|---|---|---|---|---|---|---|---|---|---|
| | Cohort 1 | Cohort 2 | Cohort 3 | Cohort 4 | Cohort 5 | Cohort 6 | Cohort 7 | Cohort 8 | | |
| | (50 mg | (10 mg | 100 mg | (200 mg | (150 mg | (50 mg | (100 mg | (75 mg | | |
| | QD) | QD) | QD) | QD) | QD) | BID) | BID) | BID) | Average | Placebo |
| N | 6 | 6 | 6 | 6 | 6 | 6 | 6 | 6 | 6 | 16 |
| Age, Mean | 37.2 (8.2) | 31.3 (10.7) | 35 (9.8) | 28.8 (31.7) | 31 (11.5) | 31.7 (4.9) | 34.8 (9.8) | 44 (8.7) | 34.2 (9.4) | 40.1 (12.3) |
| (SD) | | | | | | | | | | |
| % Female | 66.7 | 33.3 | 33.3 | 33.3 | 33.3 | 0 | 16.7 | 33.3 | 31.2 | 25 |
| % Asian | 0 | 0 | 0 | 0 | 0 | 0 | 0 | 0 | 0 | 0 |
| % Black or | 33.3 | 50 | 66.7 | 100 | 83.3 | 66.7 | 83.3 | 33.3 | 68 | 55.3 |
| African | | | | | | | | | | |
| American | | | | | | | | | | |
| % White | 50 | 50 | 33.3 | 0 | 16.7 | 33.3 | 16.7 | 66.7 | 33 | 43.8 |
| BMI (kg/m2), | 25.7 (3.2) | 23.9 (3.1) | 25.3 (4.2) | 24.2 (3.3) | 24.3 (2.1) | 26 (2.5) | 22.8 (3.1) | 24.1 (2) | 24.5 (2.9) | 25.1 (2.4) |
| Mean (SD) | | | | | | | | | | |

TABLE 2-B
Summary of PK parameters from single administration
of LB-102 (mean (SD))

| | 10 mg | 50 mg | 100 mg | 150 mg | 200 mg |
|---|---|---|---|---|---|
| Cₘₐₓ | 24.1 | 176 | 348.2 | 596.5 | 975.7 |
| (ng/mL) | (10.7) | (52.8) | (141.8) | (117.5) | (254) |
| Tₘₐₓ (h) | 3 | 3 | 3 | 3 | 3 |
| T_{1/2} (h) | 13.7 (3.9) | 11.9 (1.8) | 14.1 (4.0) | 12.0 (1.9) | 13.0 (3.6) |
| AUC_{0-inf} | 252.6 | 1595.9 | 2809.8 | 4636.6 | 7002.1 |
| (ngh/mL) | (69.9) | (189.2) | (477.8) | (745.7) | (820.7) |
| CL/F (L/h) | 42.4 (12.6) | 31.7 (3.8) | 36.6 (6.9) | 33.1 (5.3) | 28.9 (3.4) |

TABLE 2-C
Summary of PK parameters from multiple administrations of LB-102
(mean (SD)), wherein the human subjects were administered
with the desired dose every 12 hours.

| | 50 mg | 75 mg | 100 mg |
|---|---|---|---|
| Cₘₐₓ (ng/mL) D1 | 125.5 (22.7) | 267.3 (69.4) | 325.2 (67.7) |
| Tₘₐₓ (h) D1 | 2.5 | 3 | 2.5 |
| T_{1/2} (h) D1 | 4.2 (0.4) | 4.0 (0.5) | 4.12 (0.6) |
| AUC_{0-inf} (ngh/mL) D1 | 1012.6 (100.4) | 1777.9 (309.0) | 2152.9 (439.9) |
| Cₘₐₓ (ng/mL) D7 | 224.0 (39.9) | 309.4 (149.1) | |
| Tₘₐₓ (h) D7 | 2.5 | 2 | |
| T_{1/2} (h) D7 | 14.3 (3.7) | | |
| AUC_{0-inf} (ngh/mL) D7 | 2489.1 (312.4) | | |
| CL/F (L/h) | 33.8 (3.1) | 34.9 (8.8) | |

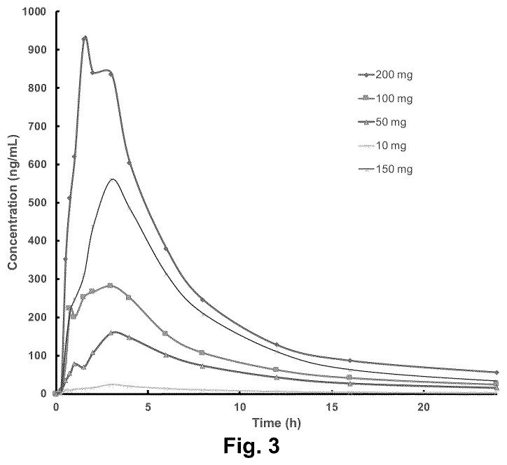

Plasma concentrations of LB-102 were obtained as a function of time for single oral administration of LB-102 to human subjects at 10 mg (larger cross), 50 mg (triangle), 100 mg (square), 150 mg (smaller cross), and 200 mg (diamond), respectively, are further provided in FIG. 3.

PK profile of a single oral administration of LB-102 (50 mg) to human subjects (FIG. 4B) showed an AUC (1,595 ngh/mL) that was about 2.5 times of the AUC (603 ngh/mL) obtained from PK profile of an administration of 50 mg amisulpride previously published (FIG. 4A).

Orally dosed LB-102 was rapidly absorbed and exposure increased in a slightly greater than dose proportional manner. In the MAD portion of the study trough concentrations of LB-102 plateaued prior to the morning dose on Day 4 and showed a slight to moderate accumulation of across dose levels.

Plasma exposure of LB-102 was markedly above expectations compared to animal models and published data on amisulpride.

### Example 3: Dopamine (e.g., D₂/D₃) RO and PK Analysis of Administration of LB-102 to Healthy Human Subjects with Various Dosage Regimens

Healthy subjects were dosed orally with LB102 of 50 mg QD (n=4), 75 mg QD (n=4), 100 mg QD (n=4), 50 mg SS (n=2), or 100 mg SS (n=2). Subjects were, on average, 33 years old. Subjects dosed QD were given a single dose on day 1 after which PET scans were obtained; and subjects dosed SS, i.e., steady state, were dosed once a day for 4 days and PET scans were obtained after dosing on Day 4.

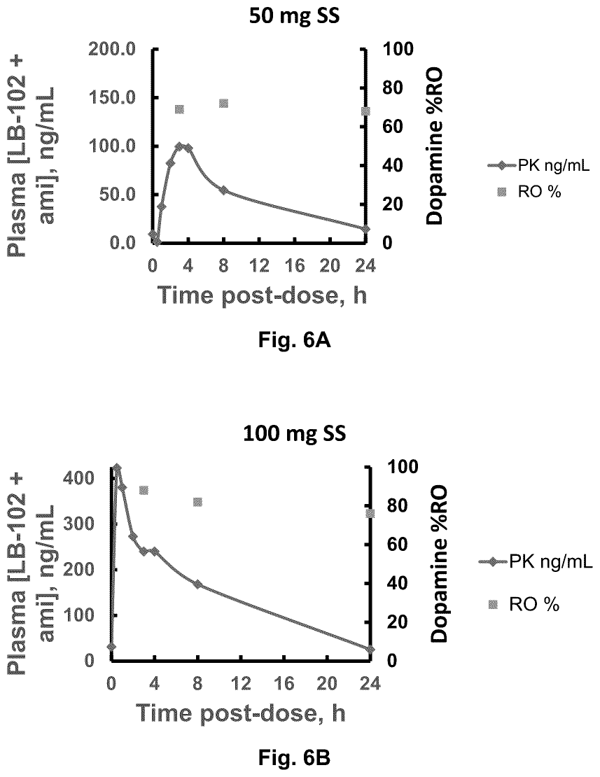

Dynamic 110 raclopride PET scans were obtained 0 h, 2.5 h, 7.5 h, and 23.5 h after LB-102 administration for the 50 mg and 100 mg QD treatment groups and after LB-102 administration of Day 4 for the 50 mg SS and 100 mg SS treatment groups; and 0 h, 3.5 h, 23.5 h, and 47.5 h after LB-102 administration for the 75 mg QD treatment group. Dopamine % RO were calculated using the STRM method (https://pubmed.ncbi.nlm.nih.gov/9345505/, which is incorporated herein by reference) and the combined RO % (average of caudate and putamen RO %) are shown as squares FIGS. 5A (50 mg QD), 5B (75 mg QD), 5C (100 mg QD); and FIGS. 6A (50 mg SS Day 4) and 6B (100 mg SS Day 4); and summarized in Table 3-A below.

Combined plasma concentration of LB-102 and amisulpride were obtained and shown as diamond in FIGS. 5A (50 mg QD), 5B (75 mg QD), 5C (100 mg QD); and FIGS. 6A (50 mg SS Day 4) and 6B (100 mg SS Day 4); and selected data summarized in Table 3-A below.

Typically dopamine RO resulting from treatment with dopamine antagonists closely tracks the corresponding plasma concentration, e.g., brexipiprazsole [D. F. Wong, A. Raufinia, P. Bricmont, J. R. Brasic, R. D. McQuade, R. A. Forbes, T. Kikuchi, and H. Kuwabara, “An open-label, positron emission tomography study of the striatal D2/D3 receptor occupancy and pharmacokinetics of single-dose oral brexpiprazole in healthy participants,” European Journal of Clinical Pharmacology, 2021, 77, 717-725.), lumateperone [R. E. Davis, K. E. Vanover, Y. Zhou, J. R. Brasic, M. Buevara, B. Bisuna, W. Ye, V. Raymont, W. Willis, A. Kumar, L. Gapasin, R. R. Goldwater, S. Mates, and D. F. Wong, “ITI-007 demonstrates brain occupancy at serotonin 5-HT2A and dopamine D2 receptors and serotonin transporters using positron emission tomography in healthy volunteers”, Psychopharmacology, 2015, 232, 2863-2872.], and ziprasidone [I. Vernalekan, C. Fellows, H. Janouschek, A. Brocheler, T. Veselinovic, C. Landvogt, C. Boy, H.-G. Buchholz, K. Sprecklmeyer, P. Bartenstein, P. Cumming, C. Hiemke, F. Rosch, W. Schafer, D. F. Wong, and G. Grunder, “Striatal and Extrastriatal D2/D3-Receptor—Binding Properties of Ziprasidone A Positron Emission Tomography Study With [18F]Fallypride and [11C]Raclopride (D2/D3-Receptor Occupancy of Ziprasidone)”, J. Clin. Psychopharmacol., 2008, 28, 608-617.].

Unexpectedly, dopamine RO of LB-102 persisted significantly after the combined plasma concentration of LB-102 and amisulpride dropped sub 10 ng/mL.

TABLE 3-A
Combined Dopamine % RO (Average of Caudate + Putamen) and
Combined Plasma concentration of LB-102 and amisulpride after LB-102
administration to human subjects, the mean values of each group of subjects were
provided.

| | Time after LB-102 administration | 0 h | 3 h | 8 h | 24 h | 48 h |
|---|---|---|---|---|---|---|
| 50 mg QD | Plasma [LB-102 + amisulpride] (ng/ml) | 0.0 | 98 | 64.6 | 11.8 | N/A |
| | Dopamine RO % (Caudate + Putamen) | 0 | 24 | 38 | 42 | N/A |
| 75 mg QD | Plasma [LB-102 + amisulpride] (ng/ml) | 0.0 | 184 | 119 | 21 | 6 |
| | Dopamine RO % (Caudate + Putamen) | 0 | 33 | N/A | 55 | 37 |
| 100 mg QD | Plasma [LB-102 + amisulpride] (ng/ml) | 0.0 | 313 | 199 | 30.4 | N/A |
| | Dopamine RO % (Caudate + Putamen) | 0 | 41 | 70 | 69 | N/A |
| 50 mg SS | Plasma [LB-102 + amisulpride] (ng/ml) | 9.1 | 99.4 | 54.5 | 14.4 | N/A |
| | Dopamine RO % (Caudate + Putamen) | N/A | 69 | 72 | 68 | N/A |
| 100 mg SS | Plasma [LB-102 + amisulpride] (ng/ml) | 31 | 290 | 168 | 25 | N/A |
| | Dopamine RO % (Caudate + Putamen) | N/A | 88 | 82 | 76 | N/A |

Dynamic ¹¹C raclopride PET scan results for calculation of dopamine RO % were measured in caudate, putamen, thalamus, and temporal lobe of each subject tested, and are summarized in Tables 3-B to 3-F below.

TABLE 3-B
Dopamine RO % after LB-102 administration to human subjects (50 mg QD)

| | Subject 1 | | | Subject 2 | | | Subject 3 | | | Subject 4 | | |
|---|---|---|---|---|---|---|---|---|---|---|---|---|
| | Time after LB-102 admin. (h) | | | | | | | | | | | |
| | 2.5 | 7.5 | 23.5 | 2.5 | 7.5 | 23.5 | 2.5 | 7.5 | 23.5 | 2.5 | 7.5 | 23.5 |
| Caudate | 26 | 52 | 58 | 28 | 42 | 45 | N/A | 36 | 53 | 23 | 43 | 45 |
| Putamen | 23 | 47 | 48 | 25 | 37 | 36 | N/A | 25 | 37 | 19 | 34 | 34 |
| Thalamus | 40 | 48 | 39 | 42 | 31 | 23 | N/A | 42 | 45 | 26 | 28 | 18 |
| Temporal lobe | 25 | 32 | 30 | 16 | 14 | 11 | N/A | 20 | 18 | 23 | 43 | 45 |

TABLE 3-C
Dopamine RO % after LB-102
administration to human subjects (75 mg QD)

| | Subject 1 | | | Subject 2 | | | Subject 3 | | | Subject 4 | | |
|---|---|---|---|---|---|---|---|---|---|---|---|---|
| | Time after LB-102 admin. (h) | | | | | | | | | | | |
| | 3.5 | 23.5 | 47.5 | 3.5 | 23.5 | 47.5 | 3.5 | 23.5 | 47.5 | 3.5 | 23.5 | 47.5 |
| Caudate | 27 | 70 | 53 | 53 | 58 | 45 | 57 | 69 | 52 | 5 | 48 | 13 |
| Putamen | 20 | 52 | 38 | 48 | 53 | 39 | 50 | 53 | 38 | 2 | 37 | 12 |
| Thalamus | 31 | 64 | 16 | 44 | 37 | 25 | 37 | 36 | 20 | 5 | 21 | −25 |
| Temporal | 12 | 45 | 4 | 21 | 17 | 16 | 28 | 20 | 16 | −2 | 27 | −6 |
| lobe | | | | | | | | | | | | |

TABLE 3-D
Dopamine RO % after LB-102 administration to human subjects (100 mg QD)

| | Subject 1 | | | Subject 2 | | | Subject 3 | | | Subject 4 | | |
|---|---|---|---|---|---|---|---|---|---|---|---|---|
| | Time after LB-102 admin. (h) | | | | | | | | | | | |
| | 2.5 | 7.5 | 23.5 | 2.5 | 7.5 | 23.5 | 2.5 | 7.5 | 23.5 | 2.5 | 7.5 | 23.5 |
| Caudate | 39 | 75 | 76 | 56 | 78 | 72 | 50 | 84 | 76 | 26 | 55 | 68 |
| Putamen | 32 | 67 | 68 | 52 | 75 | 66 | 46 | 77 | 69 | 25 | 49 | 56 |
| Thalamus | 28 | 40 | 26 | 42 | 49 | 43 | 34 | 46 | 39 | 31 | 49 | 39 |
| Temporal lobe | 18 | 24 | 13 | 17 | 17 | 22 | 20 | 24 | 22 | 10 | 15 | 15 |

TABLE 3-E
Dopamine RO % after LB-102 administration to
human subjects (50 mg SS)

| | Subject 1 | | | Subject 2 | | |
|---|---|---|---|---|---|---|
| Time after LB-102 admin. (h) | ~3 | ~8 | ~24 | ~3 | ~8 | ~24 |
| Caudate | 75 | 78 | 75 | 74 | 76 | 73 |
| Putamen | 67 | 69 | 66 | 66 | 70 | 63 |
| Thalamus | 44 | 40 | 30 | 37 | 43 | 56 |
| Temporal lobe | 18 | 17 | 35 | 26 | 25 | 34 |

TABLE 3-F
Dopamine RO % after LB-102 administration to
human subjects (100 mg SS)

| | Subject 3 | | | Subject 4 | | |
|---|---|---|---|---|---|---|
| Time after LB-102 admin. (h) | ~3 | ~8 | ~24 | ~3 | ~8 | ~24 |
| Caudate | 84 | 86 | 80 | 88 | 89 | 83 |
| Putamen | 80 | 83 | 73 | 79 | 80 | 72 |
| Thalamus | 50 | 51 | 48 | 78 | 80 | 69 |
| Temporal lobe | 19 | 18 | 23 | 32 | 25 | 31 |

### REFERENCES

The references listed below, and all references cited in the specification are hereby incorporated by reference in their entireties, as if fully set forth herein.
1) H. Y. Meltzer and S. S. Stahl, “The Dopamine Hypothesis of Schizophrenia—A Review,” Schizophr. Bull., 1976, 2, 19-76.
2) J. J. Joyce and J. H. Meador-Woodruff, “Linking the Family of D2 Receptors to Neuronal Circuits in Human Brain: Insights into Schizophrenia,” Neuropsychopharmacology, 1997, 16, 1444-1449.
3) S. Wulff, L. Hageman Pinborg, C. Svarer, L. Thorbjom Jensen, M. Odegaard Nielsen, P. Allerup, N. Bak, H. Rasmussen, E. Frandsen, E. Rostrup, and B. Yding Glenthoj, “Striatal D2/3 Binding Potential Values in Drug-Naïve First-Episode Schizophrenia Patients Correlate with Treatment Outcome,” Schizophrenia Bulletin, 2015, 41, 1143-1152.
4) B. L. Roth, D. J. Sheffler, and W. K. Kroeze, “Magic Shotguns Versus Magic Bullets: Selectively Non-Selective Drugs for Mood Disorders and Schizophrenia,” Nature Reviews Drug Discovery, 2004, 3, 353-359.
5) M. Thominet, J. Acher, and J.-C. Monier, “Derivatives of 4-Amino-5-Alkyl Sulphonyl Orthoamides,” U.S. Pat. No. 4,401,822, Filed Oct. 9, 1981 (Issued Aug. 30, 1983).
6) H. Shoemaker, Y. Claustre, D. Fage, L. Rouquier, K. Chergui, 0. Curet, A. Oblin, F. Gonon, J. Benavides, and B. Scatton, “Neurochemical Characteristics of Amisulpride, An Atypical Dopamine D2/D3 Receptor Antagonist with Both Presynaptic and Limbic Selectivity,” J. Pharmacol. Exp. Ther., 1997, 280, 83-97.
7) A. A. Abbas, P. B. Hedlund, X-P. Huang, T. B. Tran, H. Y. Meltzer, and B. L. Roth, “Amisulpride Is a Potent 5-Ht7 Antagonist: Relevance for Antidepressant Actions In Vivo,” Psychopharmacology, 2009, 119-128.
8) S. Jafari, F. Fernandez-Enright, and X.-F. Huang, “Structural Contributions of Antipsychotic Drugs to Their Therapeutic Profiles and Metabolic Side Effects,” J. Neurochemistry, 2012, 120, 371-384.
9) J. N. Dos Santos Pereira, S. Tadjerpisheh, M. Abu Abed, A. R. Saadatmand, B. Weksler, I. A. Romero, P.-O. Couraud, J. Brockmoller, and M. V. Tzvetkov, “The Poorly Membrane Permeable Antipsychotic Drugs Amisulpride and Sulpiride Are Substrates of the Organic Cation Transporters from the SLC22 Family,” The AAPS Journal, 2014, 16, 1247-1258.
10) J. C. Neill, S. Barnes, S. Cook, B. Grayson, N. F. Idris, S. L. McLean, S. Snigdha, L. Rajagopal, and M. K. Harte, “Animal Models of Cognitive Dysfunction and Negative Symptoms of Schizophrenia: Focus on NMDA Receptor Antagonism,” Pharmacology & Therapeutics, 2010, 128, 419-432.
11) J. C. Neill, M. K. Harte, P. M. Haddad, E. S. Lydall, and D. M. Dwyer, “Acute and Chronic Effects of Nmda Receptor Antagonists in Rodents, Relevance to Negative Symptoms of Schizophrenia: A Translational Link to Humans,” European Neuropsychopharmacology, 2014, 24, 822-835.
12) J. C. Neill, B. Grayson, B. Kiss, I. Gyertyan, P. Ferguson, and N. Adham, “Effects of Cariprazine, A Novel Antipsychotic, On Cognitive Deficit and Negative Symptoms in a Rodent Model of Schizophrenia Symptomatology,” European Neuropsychopharmacology, 2016, 26, 3-14.

We claim:

1. A method for delivering a dopamine and/or serotonin (e.g., 5-HT2a, 5-HT₇) and/or alpha-2 adrenergic (α2) receptor antagonist to the brain of a subject comprising administering to the subject a therapeutically effective amount of an amisulpride derivative disclosed herein or a pharmaceutical composition thereof.

2. A method for antagonizing dopamine and/or serotonin (e.g., 5-HT2a, 5-HT₇) and/or α2 receptor in a subject comprising administering to a subject a therapeutically effective amount of an amisulpride derivative disclosed herein or pharmaceutical compositions thereof, individually or in combination with other CNS active agents.

3. A method for treating one or more conditions responsive to modulation of dopamine and/or serotonin (e.g., 5-HT2a, 5-HT₇) and/or α2 receptor in a subject comprising administering to a subject a therapeutically effective amount of an amisulpride derivative disclosed herein or pharmaceutical compositions thereof, either individually or in combination with other CNS active agents.

4. The method of claim 1, wherein the amisulpride derivative is LB-102.

5. The method of claim 1, wherein the dosage of the amisulpride derivative is one, two, three, or four unit doses of the amisulpride derivative.

6. The method of claim 1, wherein the amisulpride derivative is administered once a day, once every two days, once every three days, once every four days, once every five days, once every six days, or once a week.

7. The method of claim 1, wherein the unit dose is 50 mg, 75 mg, or 100 mg.

8. The method of claim 1, further comprising adjusting the dose of the amisulpride derivative to accomplish a striatal dopamine RO % or an average dopamine (e.g., D₂/D₃) RO % of caudate and putamen measured from a treated subject to about 60% to about 80%, about 50% to about 85%, or about 40% to about 90%.

9. The method of claim 1, comprising:
a) administering a first unit dose of the amisulpride derivative to the subject once a day for one day, two days, three days, four days, five days, six days, or a week;
b) obtaining a first average dopamine RO % of caudate and putamen of the subject;
c) administering to the subject a second dose of the amisulpride derivative once a day for one day, two days, three days, four days, five days, six days, or a week if a first striatal dopamine RO % or a first average dopamine (e.g., D₂/D₃) RO % of caudate and putamen of the subject is outside of a predetermined range of about 60% to about 80%, about 50% to about 85%, or about 40% to about 90%;
d) obtaining a second striatal dopamine RO % or a second average dopamine (e.g., D₂/D₃) RO % of caudate and putamen of the subject; and
e) repeat steps c) and d) until the striatal dopamine RO % or average dopamine (e.g., D₂/D₃) RO % of caudate and putamen of the subject falls within the predetermined range (e.g., about 60% to about 80%, about 50% to about 85%, or about 40% to about 90%).
